(19) United States
(12) Plant Patent Application Publication
(10) Pub. No.: US 2021/0307226 P1
(43) Pub. Date: Sep. 30, 2021

(54) Cannabis Plant Variety Named MOC20P01

(50) ;

(71) Applicant: Aurora Cannabis Enterprises Inc., Edmonton (CA)

(72) Inventors: Andrew Hand, Comox (CA); Alan Graziano, Toronto (CA)

(73) Assignee: Aurora Cannabis Enterprises Inc., Edmonton (CA)

(21) Appl. No.: 17/214,694

(22) Filed: Mar. 26, 2021

(30) Foreign Application Priority Data
Mar. 27, 2020 (CA) 20-10130

(51) Int. Cl. A01H 6/28

(52) U.S. Cl. PLT/258

(57) ABSTRACT

A new cultivar of Cannabis plant named MOC20P01 that is characterized by about 1% THC and about 16% CBD by dry weight.

Genus and species designation: Cannabis sativa.

Variety denomination: The varietal denomination of the claimed Cannabis (Cannabis sativa) cultivar is MOC20P01.

### CROSS-REFERENCE TO RELATED APPLICATION

This application claims the benefit of priority under 35 U.S.C. 199(f) to Canadian Plant Breeders' Rights Application Number 20-10130, which was filed for the instant plant variety on Mar. 27, 2020.

### BACKGROUND

The present disclosure relates to a new and distinct Cannabis (Cannabis sativa) cultivar designated as MOC20P01.

MOC20P01 is a selection resulting from a controlled cross of female Cannabis sativa cultivar Nollia (Buddha's Sister Seed#35, MOB19P01) and male Cannabis sativa cultivar of uncertain background referred to as Med #1 Seed #1 (no agricultural ID). The plants were grown in an indoor Cannabis facility in Markham, Ontario, Canada. The plants were flowered under standard indoor environment conditions with High Pressure Sodium (HPS) and light-emitting diode (LED) lamps with automatic ebb and flow irrigation.

Seeds from the cross were sown in the same facility in Markham, Ontario, Canada, and plants were screened for a number of traits including tetrahydrocannabinol (THC) and cannabidiol (CBD) levels, yield, flowering time, disease resistance, flower morphology, consistency, terpene content, and aroma.

In March 2018, MOC20P01 was first asexually propagated by apical stem cuttings approximately 10 cm long and having multiple auxiliary meristems, in Markham, Ontario, Canada. MOC20P01 is stable and reproduces true to type in successive generations of asexual reproduction. This cultivar produces high CBD levels, an increased CBD to THC ratio, which permits a relatively high production and extraction efficiency for CBD from the a given growing environment.

### SUMMARY

This invention relates to a new and distinctive Cannabis cultivar designated as MOC20P01.

MOC20P01 exhibits about 1% THC and 16% CBD by dry weight, a lack of observed disease issues, consistent batch characteristics, which ultimately permits highly efficient CBD production and extraction at scale.

### DESCRIPTION OF THE DRAWINGS

The accompanying color photographs depict characteristics of MOC20P01. The colors shown are as true as can be reasonably obtained by conventional photographic procedures.

FIG. 1 shows a whole plant clone of MOC20P01.

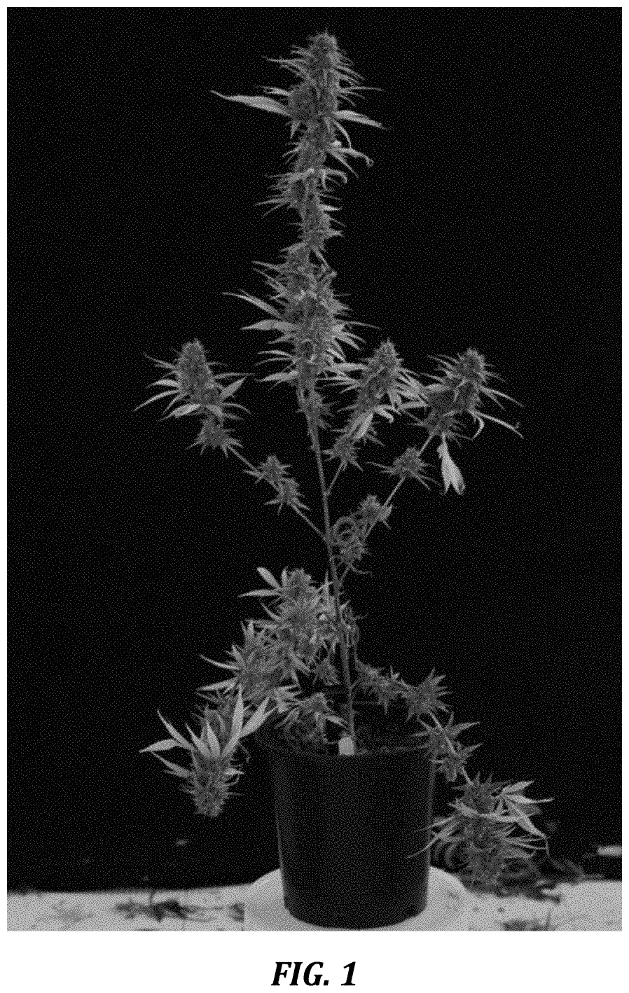

FIG. 2 shows a medium view of the stem, branches, inflorescence, and foliage of MOC20P01.

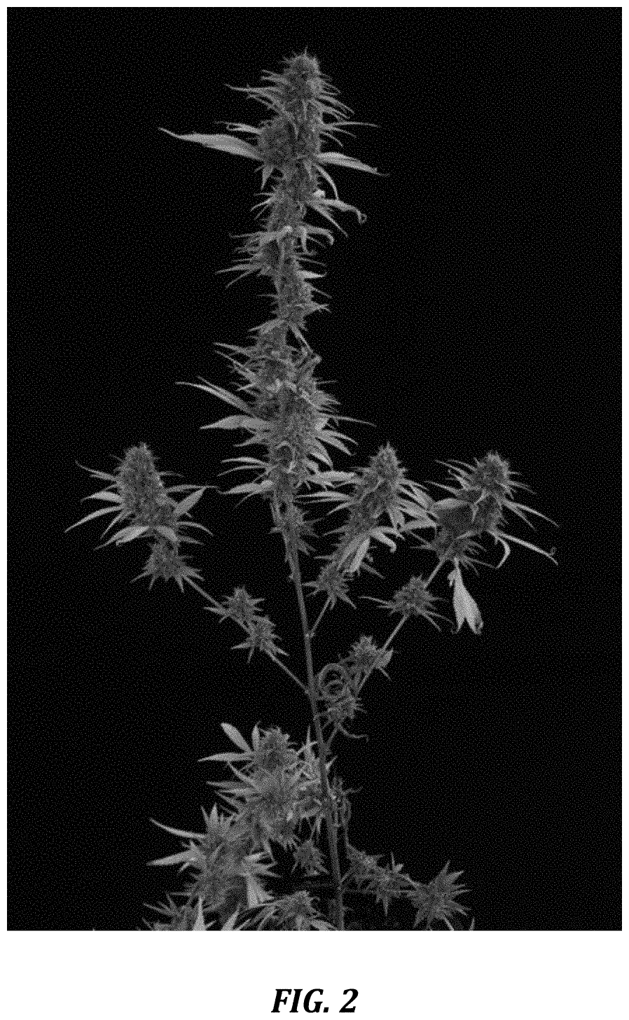

FIG. 3 shows an alternative medium view of the stem, branches, inflorescence, and foliage of MOC20P01.

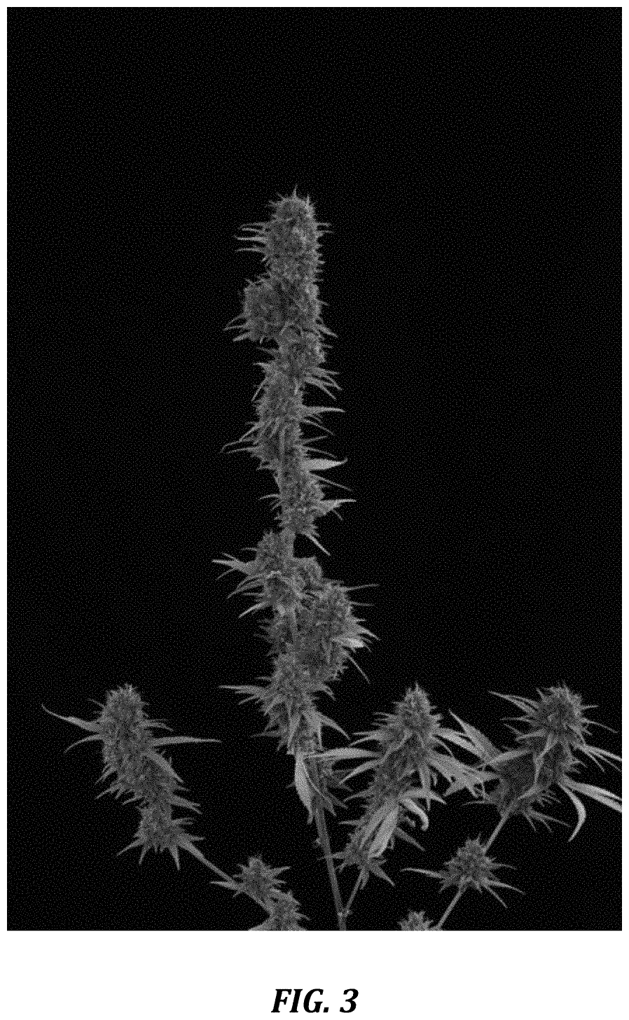

FIG. 4 shows a close-up view of an inflorescence and foliage of MOC20P01.

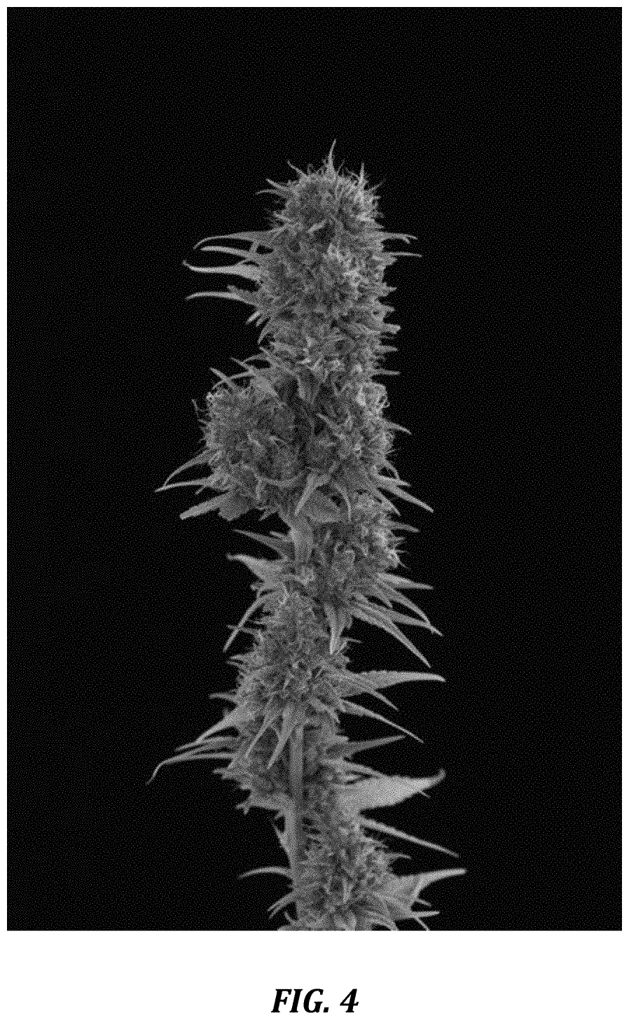

FIG. 5 shows a close-up view of a bud of MOC20P01.

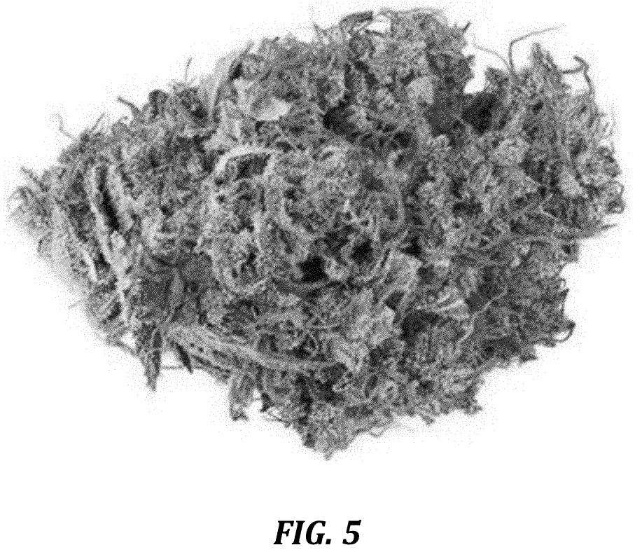

### DETAILED BOTANICAL DESCRIPTION

The following is a detailed description of the new cultivar MOC20P01.

MOC20P01 has not been tested under all possible environmental conditions. Phenotypic differences may be observed with variations in environment without any variance in genotype.

The traits of MOC20P01 have been repeatedly observed and represent the distinguishing characteristics of MOC20P01.

The data that follows was collected in Markham, Ontario Canada.

Plants were flowered under standard indoor environmental conditions with High Pressure Sodium (HPS) and light-emitting diode (LED) lamps. Clones were cut from healthy mothers and allowed to root for 14 to 18 days prior to being introduced to the flower room. The room was maintained at 18 hour days/6 hour nights for a week to allow the cuttings to establish adequate vegetative tissue. The floral transition was initiated by switching to 12 hour days and 12 hour nights. Flowering lasted 8-9 weeks before harvest, at which point samples were taken for chemical analysis, and yield was quantified. Morphological measurements were taken from plants aged 9 weeks post-floral transition.

Standard in-house developed nutrients were used throughout growth, and the plants were defoliated and pruned as necessary throughout the cycle.

In the following description, the color determination is in accordance with the 2017 Munsell Plant Tissue Color Book, except where general color terms of ordinary dictionary significance are used.
Classification:
   Denomination.—MOC20P01.
   Family.—Cannabacaeae.
   Genus.—Cannabis.
   Species.—Cannabis sativa.
   Common name.—Marijuana.
Parentage: Female Cannabis sativa variety “Nollia” (Buddha's Sister Seed#35, MOB19P01) and male Cannabis sativa variety of uncertain background referred to as “Med #1 Seed #1” (no agricultural ID).
Propagation: MOC20P01 is asexually (clonally) propagated from vegetative cuttings. It takes approximately 14-18 days to produce a rooted young plant. Roots are fine and well-branched.
Plant:
   Height.—79.5 cm-93.5 cm (average 86.5 cm).
   Diameter.—35 cm-60 cm.
Stems:
   Length/height.—79.5 cm-93.5 cm (average 86.5 cm).
   Width/diameter.—8 mm-10 mm.
   Color.—2.5 GY 5/6 [e.g., 2.5 GY 7/8, 5 RP 3/4].
   Shape.—Tubular.
   Texture.—Fibrous and Pubescent.
   Nodes.—21-24 (average 23).
Foliage:
   Type/form.—Palmately Compound.
   Arrangement.—Alternate.
   Attachment.—Petiolate.
   Leaf width.—17 cm-20 cm.
   Leaf length.—14 cm-16 cm.
   Number of leaflets per leaf.—5-7.
   Leaflet shape.—Lanceolate.
   Leaflet length.—14 cm-16 cm.
   Leaflet width.—2.5 cm-3 cm.
   Leaflet margin.—Serrate.
   Leaflet apex.—Acuminate.
   Leaflet base.—Attenuate.
   Leaflet color, upper surface.—7.5 GY 3/4.
   Leaflet color, lower surface.—5 GY 5/4.
   Venation pattern and description, upper and lower leaflet surfaces.—Pinnate.
   Texture (both surfaces).—Pubescent, primarily on the abaxial side and along leaf vasculature.
   Stipules.—Present, 2 per node on either side of a petiole, attenuated.
   Fragrance.—Earthy, herbal, tea aroma.
Petiole:
   Length.—4 cm-6 cm.
   Diameter.—˜2 mm.
   Texture.—Fine, short non-glandular trichomes.
   Color.—5 GY 6/6, 5 RP 3/6.
Inflorescence: The plant is a genetically female dioicous plant and, therefore, produces predominantly female flowers. There are no male-only plants. There is a very low incidence of hermaphroditism.
   Blooming habit.—Short day photo period photoperiod sensitive.
   Blooming period.—56 days (average) from photoperiod switch. Male flowering: male flowers are very rare and have not been observed.
   Attachment.—Subsessile.
   Bracts.—Covered with trichomes and resin glands with 2 stigmas.
   Bract color.—5GY 6/10 (exterior) and 5GY 5/6 (interior).
   Trichomes.—Capitate-sessile, capitate-stalked, bulbous, and non-glandular (cystolith hairs); trichomes are present on almost all aerial organs but are present at highest concentration on female flowers (calyxes, bracts, etc.) as well as subtending intra-flower leaves.
   Bract trichome, resin gland color.—Mix of mix of clear, cloudy, and 10R 4/8.
   Inflorescence color.—5 GY 4/4.
   Diameter.—2.5 cm-5 cm.
   Length.—3 cm-7 cm.
   Inflorescence anthocyanin.—Absent for both female and male (very rare) flowers.
   Average flower width.—4.4 cm.
   Inflorescence fragrance.—Earthy, herbal, tea aroma.
Reproductive organs:
   Pistils.—1.
   Quantity (of stigmas) per flower.—2-2.
   Stigma length.—0.3 cm-0.6 cm.
   Stigma width.—<1 mm.
   Stigma color.—2.5 GY 8/4, 7.5 YR 5/6, 10 R 3/4.
THC and CBD content: (as quantified with a Waters LC-MS/MS, running an Acetonitrile:Methanol:2-propanol gradient mobile phase through a Raptor ACR-18, 2.7 um, 2.1×150 mm column):
   Average % THC by dry weight.—1%.
   Average % CBD by dry weight.—13%-18% (average 16.2%).
Yield:
   Average flower yield.—810 g/m2 (grams dried, trimmed flower per m2 of growing space).
Disease and insect/pest resistance: Not directly tested. However, no disease or pest issues have been observed.
Comparison with known varieties: When compared to parental cultivar Nollia (CBD: 5-8%; THC (3-5%), MOC20P01 produces higher CBD and lower THC. MOC20P01 also yields significantly more mass than Nollia (average of 378 g/m²). Nollia is also shorter than MOC20P01 at 70 cm on average. A close variety of Cannabis known to the inventors is the commercial variety “Avidekel”. The variety MOC20P01 can be distinguished from the variety “Avidekel” by the traits shown in Table 1.

TABLE 1
Comparison between MOC20P01 and Avidekel

| Characteristic | MOC20P01 | Avidekel |
|---|---|---|
| Overage Average Height | 86.5 cm | 49 cm |
| Average Active Height | 80.7 cm | 39 cm |
| Average # of Nodes | 23 | 26 |
| Average Yield (g/m2) | 819.1 | 551 |
| Average Flower Width | 4.4 cm | 3.7 cm |
| Average % THC by Dry Weight | 1% | 0.55% |
| Average % CBD by Dry Weight | 13-18% | 12.44% |

In comparison to the variety “Avidekel”, the MOC20P01 variety is characterized as a high CBD producing cultivar. The MOC20P01 variety provides a high yield of CBD, which increases the grams CBD/m² growing space relative to other CBD-producing cultivars, such as the “Avidekel” variety. This improvement allows for improved extraction efficiency. MOC20P01 propagates easily, grows consistently batch to batch, and had no observed disease issues to date, all of which promote production and extraction of CBD at scale.

The embodiments of the invention in which an exclusive property or privilege is claimed are defined as follows:

1. A new and distinct cultivar of Cannabis plant named ‘MR2018002’ as described and illustrated herein.
